(19) United States
(12) Patent Application Publication
(10) Pub. No.: US 2006/0096504 A1
(43) Pub. Date: May 11, 2006

(54) Adhesive bone cement

(76) Inventors: Liam Grover, Montreal (CA); Jake Edward Barralet, Montreal (CA); Adrian Jerome Wright, Birmingham (GB)

Correspondence Address: CHIEF PATENT COUNSEL;SMITH & NEPHEW, INC., 1450 BROOKS ROAD, MEMPHIS, TN, 38116, US

(21) Appl. No.: 10/529,338

(22) PCT Filed: Sep. 26, 2003
(86) PCT No.: PCT/GB03/04197

§ 371 Date: Sep. 29, 2005

(30) Foreign Application Priority Data
Sep. 26, 2002 (GB) 0222291.7

(51) Int. Cl. A61F 2/28

(52) U.S. Cl. 106/691; 106/690; 623/23.62

(57) ABSTRACT

The invention relates to bone cement compositions comprising a calcium component and a liquid component in which the liquid component comprises pyrophosphate ions with either orthophosphate ions or water, or with both orthophosphate ions and water.

The invention relates to bone adhesives and bone cements, in particular the use of novel adhesive bone cements in surgery.

There is a clinical need to fill defects in bone, for example following removal of diseased bone or trauma. These defects can be repaired using ceramic bone grafts, or alternatively using bone cements that can be moulded before setting.

The most widely used bone cements are based on polymethylmethacrylate (PMMA). These have good strength characteristics but also have a number of drawbacks: they are not adhesive to bone and release heat on curing.

Consequently ceramic bone cements have been created based on the dissolution of tetracalcium phosphate and dicalcium phosphate dihydrate (apatite cements). These have been formed with a variety of other soluble precursors to yield cements with altered properties. One such cement, developed by reacting at a lower pH, is a brushite cement. This ceramic cement is capable of being replaced by bone following implantation. However, this improvement in biocompatibility is coupled with poorer mechanical properties than apatite cements. Currently used ceramic bone cements such as brushite are brittle and may crack and/or delaminate from the bone surface when put under load.

The nucleation and growth of brushite crystals in cements is very rapid, meaning that brushite cements usually set in a period of ˜1-2 min. For this reason various additives such as pyrophosphates, sulphates and citrates have been used at low levels to retard the cement setting reaction.

There are other clinical needs that are not met by current products. For example, during reconstructive surgery it is sometimes desirable to attach together a number of bone fragments. This is not possible using currently available, non-adhesive bone cements (PMMA or ceramic).

There exists a range of adhesive methodologies that could be used to stick bone cements and fragments in place. These include biological glues such as fibrin (the polymerisation of fibrinogen to fibrin via thrombin catalysis) and synthetic glues such as cyanoacrylates. It is also possible to surgically join bones by the use of metal pins, screws and plates. However, none of these approaches adequately solves the problem.

Fixing the bone cement or fragments in place with a biological adhesive is not an appropriate solution because these glues do not have sufficient strength to cope with the stresses within bone.

Fixing the bone cement or fragments in place with a standard synthetic adhesive is not an appropriate solution because many synthetic adhesives have been manufactured for industrial and consumer uses so are not suitable for use in the body due to toxicity of adhesive ingredients, slow adhesive curing in moist conditions at body temperature and poor biodegradation of the cured adhesive.

Fixing the bone cement or bone fragments into place with metal pins, although a commonly used technique, is not an optimal solution as this causes considerably more trauma to the site and the patient.

It is an objective of the present invention to provide biocompatible, ceramic-based bone cement with appropriate strength characteristics that is adhesive that sets over a clinically relevant timescale (typically 1 to 30 minutes at room temperature).

Accordingly, to the present invention there is provided a bone cement composition comprising a calcium component and a liquid component in which the liquid component comprises a mixture of pyrophosphate ions and at least one of the following: orthophosphate ions and/or water. Thus the liquid component may comprise pyrophosphate with either orthophosphate ions or water, or with both orthophosphate ions and water.

Suitable sources of pyrophosphate ions include pyrophosphoric acid or non-toxic, soluble pyrophosphate salts, which are aptly sodium salts and suitably tetrasodium pyrophosphate, disodium dihydrogen pyrophosphate and the like. Suitable sources of orthophosphate ions include orthophosphoric acid or non-toxic, soluble orthophosphate salts, which are aptly sodium salts.

Typically the liquid component comprises between 0 and 90% water by weight. Aptly the liquid component comprises water within the range 0 to 60%, 30 to 90%, 20 to 70%, 30 to 60%.

Typically the liquid component comprises at least 10% pyrophosphoric acid or source of pyrophosphate ions by weight.

Preferably the liquid component comprises no more than 85% pyrophosphoric acid or source of pyrophosphate ions by weight.

Typically the liquid component comprises at least 5% orthophosphoric acid or source of orthophosphate ions by weight.

Preferably the liquid component comprises no more than 85% orthophosphoric acid or source of orthophosphate ions by weight.

Typically the cement comprises between 0.8 g to 3.0 g calcium component to 1 ml liquid component.

Preferably the cement comprises between 1.5 g to 2.5 g calcium component to 1 ml liquid component.

Preferably the calcium component is a solid in the 10 nm to 100 μm primary particle size range.

Aptly the calcium component is a calcium phosphate, calcium oxide or calcium hydroxide.

Typically the calcium component may be one or more of β-tricalcium phosphate (β-TCP), α-tricalcium phosphate (α-TCP), tetracalcium phosphate (TTCP), dicalcium phosphate anhydrous (DCPA), dicalcium phosphate dihydrate (DCPD), hydroxyapatite (HA) or calcium oxide (CaO).

Aptly, the adhesion properties of the cement are conferred by the liquid component of the cement where said liquid component comprises water within the range 0 to 90% by weight, pyrophosphoric acid 10 to 80% by weight and orthophosphoric acid 5 to 80% by weight.

Suitably, the strength properties of the cement are conferred by the liquid component of the cement where said liquid component comprises water within the range 30 to 80% by weight, pyrophosphoric acid 10 to 80% by weight and orthophosphoric acid 5 to 40% by weight.

Preferably, the setting properties of the cement are conferred by the liquid component of the cement where said liquid component comprises water within the range 0 to 90% by weight, pyrophosphoric acid 10 to 80% by weight and orthophosphoric acid 5 to 40% by weight.

When set the solid and liquid reactants of the bone cement, set to form a solid component which may or may not contain liquid.

A cement that combined preferable adhesive, strength and setting properties preferably contains liquid component comprising water within the range 45 to 80% by weight, pyrophosphoric acid 10 to 50% by weight and orthophosphoric acid 5 to 40% by weight.

The bone cements may comprise a retardant in order to delay the setting time. Suitable retardants include chitosan, calcium sulphate hemihydrate, trisodium citrate, sodium acetate, sodium glycolate, sodium lactate, non-toxic orthophosphate salts, phosphate buffered saline.

The aforementioned bone cements can be manufactured using skills and processes known in the art, but may involve mixing the components together with subsequent kneading on a chilled surface. Optionally applied pressure may be applied to the mixture prior to setting in order to modulate the final compressive strength of the set cement.

### EXAMPLE 1

1.5 g of β-tricalcium phosphate (Ca₃(PO₄)₂; β-TCP; Plasma-Biotal, Tideswell, UK) was added incrementally to 1 ml of solution containing a mixture of pyrophosphoric acid, orthophosphoric acid and water (proportions shown in FIG. 1). The resultant slurry was kneaded with a spatula for a period of 30 s and cast into a PTFE mould to produce cylindrical samples of 6 mm in diameter by 12 mm in height. After hardening, the samples were stored in an oven at 37° C. and 100% relative humidity for 24 h. Samples were then tested in compression at a crosshead speed of 1 mm/min. The initial and final setting times were determined using the Gilmore needles method. In order to find the adhesive tensile strength of the cement to bone, the cement pastes were applied to the surface of transversely cut defatted ovine femurs, which were subsequently bonded and stored at 37° C. for 3 h. After this time the butt joints were loaded to failure using a Universal testing machine set at a crosshead speed of 50 mm/min. The adhesive tensile strength, compressive strength and setting times for each cement are shown in FIG. 1.

### EXAMPLE 2

Water (57% by weight), pyrophosphoric acid (18% by weight) and orthophosphoric acid (22% by weight) and polyphosphoric acid (3% by weight) was mixed with β-tricalcium phosphate (powder) at 1.25 gml⁻¹ to 2.75 gml⁻¹ powder to liquid ratios with a resultant variation in setting times and compressive strength as shown in FIG. 2.

### EXAMPLE 3

Cement was produced by mixing 1.5 g of calcium phosphate precursor (α-TCP (α-Ca₃(PO₄)₂) or TTCP (Ca₄(PO₄)₂O)) with 1 ml of solution consisting of 80 wt % 400 mM phosphate buffered saline (PBS) solution (to act as a retardant), 10 wt % pyrophosphoric acid and 10 wt % orthophosphoric acid. Both mixture of the liquid and solid component as well as subsequent kneading were performed on a chilled glass slab. Setting times and compressive strengths of the cements were determined as described in Example 1. The setting times for such cements are shown in FIG. 3.

### EXAMPLE 4

The compressive strength of a ceramic material may be related to porosity by an inverse exponential relationship; therefore by reducing porosity by means of compaction during setting it is possible to improve the compressive strength of the cement. 2 g of β-TCP was mixed with 540 mg of commercially available polyphosphoric acid (51% orthophosphoric acid, 42% pyrophosphoric acid, 7% higher chain acids) and 720 mg water, the resultant slurry was kneaded with a spatula for 30 seconds and transferred into a stainless steel split mould of 8 mm in diameter. A cylindrical tool steel ram was placed into the mould and subsequently the setting cement was compacted uniaxially to pressures of up to 100 MPa. The resultant cement compressive strengths are shown in FIG. 4.

### EXAMPLE 5

The setting reaction of the polyphosphoric acid cement can be retarded using various additives including sodium salts of alpha-hydroxyacids, chitosan and calcium sulphate hemihydrate. Briefly, 540 mg of polyphosphoric acid (51% orthophosphoric acid, 42% pyrophosphoric acid, 7% higher chain acids) was combined with 720 ml of the retardant solution prior to mixture with the β-TCP at a powder to liquid ratio of 1.75 g/ml. The calcium sulphate hemihydrate was mixed with the β-TCP before mixture with the polyacid solution. The setting times were determined using the Gilmore needles method. The results are shown in FIG. 5.

### EXAMPLE 6

It is possible to produce polyphosphate cement by using solutions containing pyrophosphate ions derived from a source other than pyrophosphoric acid. For example a setting cement was produced when an equimolar mixture of TTCP (Ca₄(PO₄)₂O) and DCPA (CaHPO₄) was combined with 400 mM tetra sodium pyrophosphate solution (Na₄P₂O₇) at a powder to liquid ratio of 3.3 g/ml.

### EXAMPLE 7

Cement was made using a solution made from a polyphosphate salt by combining calcium hydroxide (Ca(OH)₂) with a 500 mM solution of sodium tripolyphosphate (Na₅P₃O₁₀) at a powder to liquid ratio of 600 mg/ml. Cement made using this particular formulation and stored in 100% relative humidity at 37° C. for 24 h prior to testing exhibited a compressive strength of ˜3.5±0.6 MPa.

[image]

[image]

1. A bone cement composition comprising a calcium component and a liquid component in which the liquid component comprises a mixture of pyrophosphate ions and at least one of the following: orthophosphate ions and/or water.

2. A bone cement composition according to claim 1 comprising between 0.8 g to 3.0 g calcium component to 1 ml liquid component.

3. A bone cement composition according to claim 1 comprising between 1.5 g to 2.5 g calcium component to 1 ml liquid component.

4. A bone cement composition according to claim 1 where the calcium component is a solid in the 10 nm to 100 μm primary particle size range.

5. A bone cement composition according to claim 1 where calcium component is a calcium phosphate.

6. A bone cement composition according to claim 1 where the calcium component comprises at least one of the following: βtricalcium phosphate (β-TCP), α-tricalcium phosphate (α-TCP), tetracalcium phosphate (TTCP), dicalcium phosphate anhydrous (DCPA), dicalcium phosphate dihydrate (DCPD), hydroxyapatite (HA) or calcium oxide (CaO).

7. A bone cement composition according to claim 1 where the source of pyrophosphate ions is pyrophosphoric acid or a sodium pyrosphosphate.

8. A bone cement composition according to claim 1 where the source of orthophosphate ions is orthophosphoric acid.

9. A bone cement composition according to claim 7 where the liquid component comprises between 0 and 90% water by weight.

10. A bone cement composition according to claim 7 where the liquid component comprises at least 10% pyrophosphoric acid or source of pyrophosphate ions by weight.

11. A bone cement composition according to claim 7 where the liquid component comprises no more than 85% pyrophosphoric acid or source of pyrophosphate ions by weight.

12. A bone cement composition according to claim 7 where the liquid component comprises at least 5% orthophosphoric acid or source of orthophosphate ions by weight.

13. A bone cement composition according to claim 7 where the liquid component comprises no more than 85% orthophosphoric acid or source of orthophosphate ions by weight.

14. A bone cement composition according to claim 7 comprising water within the range 0 to 90% by weight, pyrophosphoric acid 10 to 80% by weight and orthophosphoric acid 5 to 80% by weight.

15. A bone cement composition according to claim 7 comprising water within the range 30 to 80% by weight, pyrophosphoric acid 10 to 80% by weight and orthophosphoric acid 5 to 40% by weight.

16. A bone cement composition according to claim 7 comprising water within the range 0 to 90% by weight, pyrophosphoric acid 10 to 80% by weight and orthophosphoric acid 5 to 40% by weight.

17. A bone cement composition according to claim 7 comprising water within the range 45 to 80% by weight, pyrophosphoric acid 10 to 50% by weight and orthophosphoric acid 5 to 40% by weight.

18. A bone cement composition according to claim 1 comprising a retardant.

19. A bone cement composition according to claim 18 where the retardant comprises at least one of the following: Chitosan, calcium sulphate hemihydrate, trisodium citrate, sodium acetate, sodium glycolate, sodium lactate, non-toxic orthophosphate salts, phosphate buffered saline.

20. A process for manufacturing a bone cement of a composition according to claim 1 where the components are compacted to a pressure between 0 MPa and 100 MPa prior to setting.

21. A bone cement composition comprising a calcium component and a liquid component comprising a solution of polyphosphate salt.

22. A bone cement composition according to claim 21 where the calcium component is calcium hydroxide.

23. A bone cement composition according to claim 21 where the polyphosphate salt is sodium tripolyphosphate.
